(19) United States
(12) Patent Application Publication
(10) Pub. No.: US 2012/0184746 A1
(43) Pub. Date: Jul. 19, 2012

(54) PROCESS FOR THE PREPARATION OF LENALIDOMIDE

(75) Inventors: Munish Kapoor, Punjab (IN); Saridi Madhava Dileep Kumar, Haryana (IN); Balaguru Murugesan, Tamil Nadu (IN); Swargam Sathyanarayana, Andhra Pradesh (IN); Rajesh Kumar Thaper, Jammu and Kashmir (IN); Mohan Prasad, Haryana (IN)

(73) Assignee: RANBAXY LABORATORIES LIMITED, New Delhi, Delhi (IN)

(21) Appl. No.: 13/393,699

(22) PCT Filed: Sep. 3, 2010
(86) PCT No.: PCT/IB10/53981

§ 371 Date: Apr. 3, 2012

(30) Foreign Application Priority Data
Sep. 3, 2009 (IN) 1823/DEL/2009

(51) Int. Cl. C07D 401/04

(52) U.S. Cl. 546/200

(57) ABSTRACT

The present invention relates to a process for the preparation of lenalidomide, wherein the process comprises: reducing 3-(4-nitro-loxo-1,3-dihydro-2H-isoindol-2-yl)piperidine-2,6-dione to obtain lenalidomide.

### FIELD OF THE INVENTION

The present invention relates to process for the preparation of lenalidomide.

### BACKGROUND OF THE INVENTION

Lenalidomide is chemically described as 3-(4-amino-1-oxo-1,3-dihydro-2H-isoindol-2-yl)piperidine-2,6-dione of Formula I.

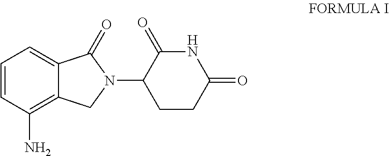

Lenalidomide is an immunomodulatory agent with antiangiogenic and antineoplastic properties. Lenalidomide is available in the market for the treatment of myelodysplastic syndromes and for the treatment of multiple myeloma.

U.S. Pat. No. 5,635,517 and WO 98/03502 both describe a process for preparing lenalidomide using the 3-(4-nitro-1-oxo-1,3-dihydro-2H-isoindol-2-yl)piperidine-2,6-dione of Formula II as an intermediate.

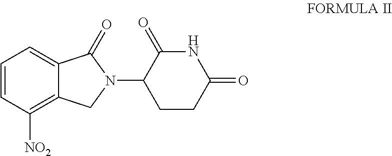

3-(4-Nitro-1-oxo-1,3-dihydro-2H-isoindol-2-yl)piperidine-2,6-dione of Formula II is prepared by reacting methyl 2-bromomethyl-3-nitrobenzoate of Formula III with 3-aminopiperidine-2,6-dione hydrochloride in the presence of N,N-dimethylformamide and triethylamine at reflux temperature for 6 hours.

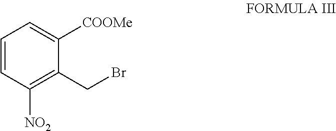

3-(4-Nitro-1-oxo-1,3-dihydro-2H-isoindol-2-yl)piperidine-2,6-dione of Formula II is reduced by hydrogenating with palladium-carbon in 1,4-dioxane at 50 psi to obtain lenalidomide.

The present inventors have observed that the conditions provided in the prior art for preparing 3-(4-nitro-1-oxo-1,3-dihydro-2H-isoindol-2-yl)piperidine-2,6-dione of Formula II, i.e., the use of N,N-dimethylformamide and triethylamine at reflux temperature, result in a black colored material, which is difficult to process, with a yield of 89%. The replacement of N,N-dimethylformamide in the prior art process with other solvents such as acetonitrile, acetone or 2-propanol still results in a black colored product with purity below 95%. Though the replacement of N,N-dimethylformamide with ethanol results in a purity of above 99%, the yield is less than 45%. Further, the present inventors have observed that the reaction requires more than 30 hours for completion.

The method provided in the prior art for reducing 3-(4-nitro-1-oxo-1,3-dihydro-2H-isoindol-2-yl)piperidine-2,6-dione of Formula II to obtain lenalidomide uses 1,4-dioxane as a solvent. However, 1,4-dioxane is used in a volume, which is 200 times higher than the weight of 3-(4-nitro-1-oxo-1,3-dihydro-2H-isoindol-2-yl)piperidine-2,6-dione of Formula II. The use of such high quantity of solvents like 1,4-dioxane is not economical on an industrial scale and is not suitable from regulatory perspective for preparing pharmaceutical substances.

While working on the above problems, the present inventors have surprisingly found that 3-(4-nitro-1-oxo-1,3-dihydro-2H-isoindol-2-yl)piperidine-2,6-dione of Formula II can be obtained with better yield, purity and quality if the reaction temperature is controlled at about 50° C. or below. Further, the reaction at about 50° C. or below can be completed within about 10 hours. 3-(4-nitro-1-oxo-1,3-dihydro-2H-isoindol-2-yl)piperidine-2,6-dione of Formula II obtained by the instant process can be easily processed in subsequent steps to obtain lenalidomide. The present inventors have also found that the reduction of 3-(4-nitro-1-oxo-1,3-dihydro-2H-isoindol-2-yl)piperidine-2,6-dione of Formula II in a solvent system comprising N,N-dimethylformamide substantially minimizes the quantity of solvent to be employed and also yields lenalidomide with a purity of about 99.8% or above. Thus, the present invention provides an efficient, industrially preferable and economic process for preparing lenalidomide.

### SUMMARY OF THE INVENTION

In one general aspect, the present invention provides a process for the preparation of 3-(4-nitro-1-oxo-1,3-dihydro-2H-isoindol-2-yl)piperidine-2,6-dione of Formula II.

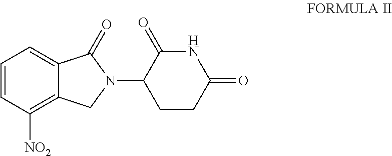

The process includes reacting methyl 2-bromomethyl-3-nitrobenzoate of Formula III

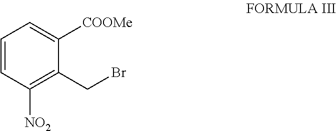

with 3-aminopiperidine-2,6-dione or its salt in the presence of an organic solvent at a temperature of about 50° C. or below to obtain 3-(4-nitro-1-oxo-1,3-dihydro-2H-isoindol-2-yl)piperidine-2,6-dione of Formula II.

In another general aspect, the present invention provides a process for the preparation of lenalidomide. The process includes:
a) reacting methyl 2-bromomethyl-3-nitrobenzoate of Formula III

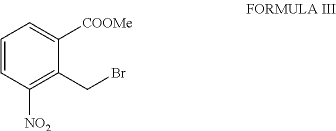

with 3-aminopiperidine-2,6-dione or its salt in the presence of an organic solvent at a temperature of about 50° C. or below to obtain 3-(4-nitro-1-oxo-1,3-dihydro-2H-isoindol-2-yl)piperidine-2,6-dione of Formula II; and

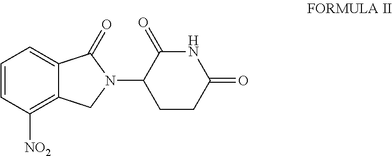

b) reducing 3-(4-nitro-1-oxo-1,3-dihydro-2H-isoindol-2-yl)piperidine-2,6-dione of Formula II to obtain lenalidomide.

Embodiments of the abovementioned aspects may include one or more of the following features. For example, the methyl 2-bromomethyl-3-nitrobenzoate of Formula III may be reacted with 3-aminopiperidine-2,6-dione or its salt at a temperature of about 20° C. to about 45° C.

The organic solvent may be a water-miscible solvent. The organic solvent may also be N,N-dimethylformamide, C₁₋₄ alkanol, C₃₋₆ ketone or acetonitrile, or a mixture thereof. The 3-(4-nitro-1-oxo-1,3-dihydro-2H-isoindol-2-yl)piperidine-2,6-dione of Formula II may have a purity of about 99.0% or above.

In another general aspect, the present invention provides a process for the preparation of lenalidomide. The process includes:
a) reducing the 3-(4-nitro-1-oxo-1,3-dihydro-2H-isoindol-2-yl)piperidine-2,6-dione of Formula II in a solvent system, which includes N,N-dimethylformamide

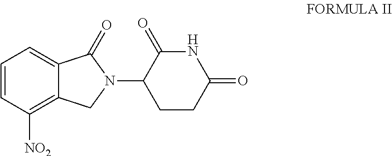

to obtain lenalidomide; and
b) isolating lenalidomide from the reaction mixture thereof.

Embodiments of this aspect may include one or more of the following features. The N,N-dimethylformamide may be used as a single solvent or in combination with one or more water-miscible organic solvents.

The water-miscible organic solvent may be methanol. The solvent may be at a volume, which is about 2 times to about 50 times more than the weight of 3-(4-nitro-1-oxo-1,3-dihydro-2H-isoindol-2-yl)piperidine-2,6-dione of Formula II. The lenalidomide produced by this aspect may have a purity of greater than about 99.8%.

### DETAILED DESCRIPTION OF THE INVENTION

A first aspect of the present invention provides a process for the preparation of 3-(4-nitro-1-oxo-1,3-dihydro-2H-isoindol-2-yl)piperidine-2,6-dione of Formula II,

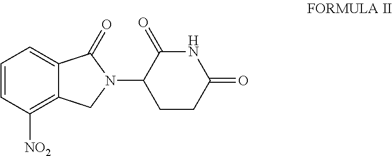

wherein the process includes a step of reacting methyl 2-bromomethyl-3-nitrobenzoate of Formula III

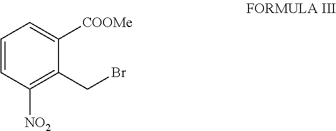

with 3-aminopiperidine-2,6-dione or its salt in the presence of an organic solvent at a temperature of about 50° C. or below to obtain 3-(4-nitro-1-oxo-1,3-dihydro-2H-isoindol-2-yl)piperidine-2,6-dione of Formula II.

A second aspect of the present invention provides a process for the preparation of lenalidomide, wherein the process includes:
a) reacting methyl 2-bromomethyl-3-nitrobenzoate of Formula III

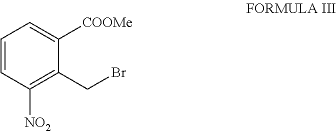

with 3-aminopiperidine-2,6-dione or its salt in the presence of an organic solvent at a temperature of about 50° C. or below to obtain 3-(4-nitro-1-oxo-1,3-dihydro-2H-isoindol-2-yl)piperidine-2,6-dione of Formula II; and

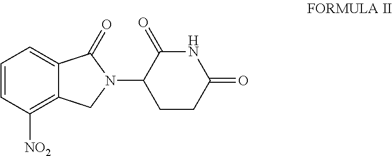

b) reducing the 3-(4-nitro-1-oxo-1,3-dihydro-2H-isoindol-2-yl)piperidine-2,6-dione of Formula II to obtain lenalidomide.

The methyl 2-bromomethyl-3-nitrobenzoate of Formula III used as a starting material may be prepared according to the method provided in WO 98/03502. Methyl 2-bromomethyl-3-nitrobenzoate of Formula III is reacted with 3-aminopiperidine-2,6-dione or its salt, for example hydrochloride salt, in the presence of an organic solvent at a temperature of about 50° C. or below, for example, from about 20° C. to about 45° C. The organic solvent may be a water-miscible solvent, for example, N,N-dimethylformamide, C₁₋₄ alkanol, C₃₋₆ ketone or acetonitrile, or a mixture thereof. The reaction may be carried out in the presence of a base. The base may be an organic or inorganic base. Alkali metal alkoxides, alkali metal hydroxides, alkali metal carbonates, alkali metal hydrides or alkylamines may be used as the base. The base may be, for example, potassium carbonate or triethylamine. The reaction may be facilitated by stirring the reaction mixture. The stirring may be carried out from about 1 hour to about 10 hours, for example, for about 2 hours to about 6 hours. The 3-(4-nitro-1-oxo-1,3-dihydro-2H-isoindol-2-yl)piperidine-2,6-dione of Formula II may optionally be isolated from the reaction mixture by filtration, precipitation, solvent evaporation, decantation, layer separation, or a combination thereof. The 3-(4-nitro-1-oxo-1,3-dihydro-2H-isoindol-2-yl)piperidine-2,6-dione of Formula II obtained has a purity of about 99.0% or above, for example, about 99.4% to about 99.9%.

The 3-(4-nitro-1-oxo-1,3-dihydro-2H-isoindol-2-yl)piperidine-2,6-dione of Formula II is further reduced to obtain lenalidomide. The reduction may be carried out in the presence of a solvent, for example, a water-miscible organic solvent. The reduction may be carried out by hydrogenating in the presence of a homogeneous or heterogeneous catalyst, or in the presence of a reducing agent. The lenalidomide obtained may be isolated from the reaction mixture by filtration, precipitation, solvent evaporation, decantation, layer separation, or a combination thereof.

A third aspect of the present invention provides a process for the preparation of lenalidomide, wherein the process includes:
a) reducing a 3-(4-nitro-1-oxo-1,3-dihydro-2H-isoindol-2-yl)piperidine-2,6-dione of Formula II in a solvent system that includes N,N-dimethylformamide

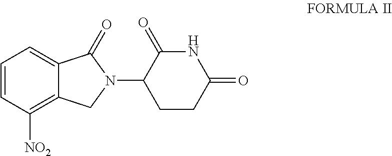

to obtain lenalidomide; and
b) isolating lenalidomide from the reaction mixture thereof.

The 3-(4-nitro-1-oxo-1,3-dihydro-2H-isoindol-2-yl)piperidine-2,6-dione of Formula II may be prepared according to the method provided in U.S. Pat. No. 5,635,517 or in the previous aspects of the present invention. The 3-(4-nitro-1-oxo-1,3-dihydro-2H-isoindol-2-yl)piperidine-2,6-dione of Formula II is reduced in a solvent system comprising N,N-dimethylformamide to obtain lenalidomide. N,N-dimethylformamide may be used as a single solvent or in combination with one or more water-miscible organic solvents. The water-miscible organic solvent may be, for example, methanol. The solvent may be used in a volume which is about 2 times to about 50 times, for example, about 8 times to about 30 times, more than the weight of 3-(4-nitro-1-oxo-1,3-dihydro-2H-isoindol-2-yl)piperidine-2,6-dione of Formula II. The reduction may be carried out by hydrogenating in the presence of a homogeneous or heterogeneous catalyst, or in the presence of a reducing agent. The reduction may be carried out, for example, by hydrogenating in the presence of palladium-carbon. The lenalidomide obtained may be isolated from the reaction mixture by filtration, precipitation, solvent evaporation, decantation, layer separation, or a combination thereof. The lenalidomide obtained has a purity of about 99.8% or above.

While the present invention has been described in terms of its specific embodiments, certain modifications and equivalents will be apparent to those skilled in the art and are intended to be included within the scope of the present invention.

### Comparative Example 1

### Preparation of 3-(4-nitro-1-oxo-1,3-dihydro-2H-isoindol-2-yl)piperidine-2,6-dione

Methyl-2-bromomethyl-3-nitrobenzoate (8.36 g) and 3-aminopiperidine-2,6-dione hydrochloride (5 g) were added to ethanol (50 ml) at 20° C. to 25° C. The temperature was raised to 50° C. to 55° C. Triethylamine (7.8 g) was added to the reaction mixture slowly over 30 minutes at 50° C. to 55° C. The reaction mixture was stirred for 32 hours at 50° C. to 55° C., cooled to 0° C. to 5° C. and stirred for 30 minutes at 0° C. to 5° C. The reaction mixture was filtered and the solid obtained was added into a mixture of dichloromethane and de-ionized water (1:2 ratio; 100 ml) at 20° C. to 25° C. The mixture was stirred for 30 minutes at 20° C. to 25° C., filtered and dried under vacuum at 50° C. to 55° C. for 17 hours to obtain the title compound.

### Yield: 41.5%

HPLC purity: 99.63%

### Comparative Example 2

### Preparation of 3-(4-nitro-1-oxo-1,3-dihydro-2H-isoindol-2-yl)piperidine-2,6-dione

Methyl-2-bromomethyl-3-nitrobenzoate (16.65 g) and 3-aminopiperidine-2,6-dione hydrochloride (10 g) were added to 2-propanol (130 ml) at 20° C. to 25° C. Triethylamine (12.3 g) was added to the reaction mixture slowly over 30 minutes at 20° C. to 25° C. The temperature of the reaction mixture was raised to 55° C. and stirred for 41 hours at 50° C. to 55° C. The reaction mixture was cooled to 20° C. to 25° C. and de-ionized water (50 ml) was added to the reaction mixture and stirred for 1 hour. The reaction mixture was filtered and the solid obtained was washed with de-ionized water (50 ml) and dried under vacuum at 45° C. to 50° C. to obtain the title compound.

### Yield: 74%

### Comparative Example 3

### Preparation of 3-(4-nitro-1-oxo-1,3-dihydro-2H-isoindol-2-yl)piperidine-2,6-dione

A mixture of 3-aminopiperidine-2,6-dione hydrochloride (50 g) and acetonitrile (650 ml) were stirred for 10 minutes at 20° C. to 25° C. Methyl-2-bromomethyl-3-nitrobenzoate (83.28 g) was added to the reaction mixture and stirred for 30 minutes. The temperature of the reaction was raised to 55° C. Triethylamine (15.37 g) was added slowly over 30 minutes at 50° C. to 55° C. and stirred for 2 hours. The above step of adding of triethylamine and stirring was repeated for three more times with same quantity, temperature and duration except that the final stirring is carried out for 40 hours at 50° C. to 55° C. The reaction mixture was cooled to 20° C. to 25° C. De-ionized water (250 ml) was added to the reaction mixture and stirred for 1 hour at 20° C. to 25° C. The reaction mixture was filtered, and the solid obtained was washed with chilled de-ionized water (100 ml) and dried under vacuum at 45° C. to 50° C. to obtain the title compound.

### Yield: 79.5%

HPLC purity: 92.28%

### Comparative Example 4

### Preparation of 3-(4-nitro-1-oxo-1,3-dihydro-2H-isoindol-2-yl)piperidine-2,6-dione

Methyl-2-bromomethyl-3-nitrobenzoate (8.38 g) and 3-aminopiperidine-2,6-dione hydrochloride (5 g) were added to N,N-dimethylformamide (50 ml) at 20° C. to 25° C. Potassium carbonate (10.5 g) was added to the reaction mixture at 20° C. to 25° C. and the temperature was raised to 55° C. to 60° C. The reaction mixture was stirred for 33 hours at 55° C. to 60° C. Approximately 20 ml of N,N-dimethylformamide was recovered under vacuum at 60° C. to 65° C. De-ionized water (50 ml) was added to the reaction mixture at 20° C. to 25° C. and stirred for 1 hour at 15° C. to 20° C. The reaction mixture was filtered, washed with de-ionized water (2×10 ml) and dried under vacuum at 50° C. to 55° C. for 18 hours to obtain the title compound.

### Yield: 26%

HPLC purity: 97.97%

### Comparative Example 5

### Preparation of 3-(4-nitro-1-oxo-1,3-dihydro-2H-isoindol-2-yl)piperidine-2,6-dione

Methyl-2-bromomethyl-3-nitrobenzoate (8.38 g) and 3-aminopiperidine-2,6-dione hydrochloride (5 g) were added to acetone (50 ml) at 20° C. to 25° C. Potassium carbonate (10.5 g) was added into the reaction mixture at 20° C. to 25° C. and the temperature was raised to 55° C. to 60° C. The reaction was stirred for 32 hours at 55° C. to 60° C. Acetone was recovered under vacuum at 55° C. to 60° C. and the residue was cooled to 20° C. to 25° C. De-ionized water (100 ml) was added to the residue and stirred for 2 hours at 20° C. to 25° C. The reaction mixture was filtered, and the solid obtained was washed with de-ionized water (2×10 ml) and dried under vacuum at 50° C. to 55° C. for 18 hours to obtain the title compound.

### Yield: 81.5%

HPLC purity: 94.02%

### Example 1

### Preparation of 3-(4-nitro-1-oxo-1,3-dihydro-2H-isoindol-2-yl)piperidine-2,6-dione

Methyl-2-bromomethyl-3-nitrobenzoate (25 g) and 3-aminopiperidine-2,6-dione hydrochloride (18 g) were added to N,N-dimethylformamide (125 ml) at 20° C. to 25° C. Potassium carbonate (31.52 g) was added to the reaction mixture at 25° C. to 30° C. and the temperature was raised to 40° C. to 45° C. The reaction mixture was stirred for 6 hours at 40° C. to 45° C. and cooled to 20° C. to 25° C. De-ionized water (125 ml) was added to the reaction mixture at 20° C. to 25° C. and stirred for 15 minutes to 20 minutes. The solid obtained was filtered, washed with de-ionized water (2×25 ml) and dried under vacuum at 40° C. to 45° C. for 20 hours to obtain the title compound.

### Yield: 91.7%

HPLC purity: 99.86%

### Example 2

### Preparation of 3-(4-nitro-1-oxo-1,3-dihydro-2H-isoindol-2-yl)piperidine-2,6-dione

3-Aminopiperidine-2,6-dione hydrochloride (25 g) and methyl-2-bromomethyl-3-nitrobenzoate (41.5 g) were added to N,N-dimethylformamide (375 ml) at 20° C. to 25° C. and stirred for 20 minutes at 20° C. to 25° C. Triethylamine (10.58 ml) was added to the reaction mixture at 20° C. to 25° C. over 5 minutes and the reaction mixture was stirred for 2 hours at 20° C. to 25° C. The above step of adding of triethylamine and stiffing was repeated for three more times with same quantity, temperature and duration. The reaction mixture was filtered, and the solid obtained was washed with de-ionized water (250 ml). The solid was stirred for 15 minutes in de-ionized water (500 ml), filtered and dried under vacuum at 45° C. to 50° C. to obtain the title compound.

### Yield: 71%

HPLC purity: 99.44%

### Example 3

### Preparation of Lenalidomide

N,N-dimethylformamide (35 ml) was added to 3-(4-nitro-1-oxo-1,3-dihydro-2H-isoindol-2-yl)piperidine-2,6-dione (5 g) at 25° C. to 30° C. in a Parr shaker hydrogenator. 10% palladium-carbon (200 mg; 50% wet) was added to the reaction mixture and the hydrogen pressure was maintained at 3 to 4 kg/cm² at 40° C. to 45° C. for 7 hours accompanied by shaking. The reaction mixture was filtered through a Celite bed and washed with N,N-dimethylformamide (10 ml). The filtrate was distilled and methanol (20 ml) was added to the solid obtained. The mixture was stirred for 14 hours at 25° C. to 30° C., filtered, washed with methanol (10 ml) and dried under vacuum at 35° C. to 40° C. for 20 hours to obtain the title compound.

### Yield: 75.8%

HPLC purity: 99.84%

### Example 4

### Preparation of Lenalidomide

N,N-dimethylformamide (500 ml) was added to 3-(4-nitro-1-oxo-1,3-dihydro-2H-isoindol-2-yl)piperidine-2,6-dione (40 g) at 25° C. to 30° C. in a Parr shaker hydrogenator followed by the addition of methanol (500 ml). 10% palladium-carbon (4 g; 50% wet) was added to the reaction mixture and the hydrogen pressure was maintained at 50 to 60 psi at 20° C. to 25° C. for 3 hours accompanied by shaking. The reaction mixture was filtered through a Celite bed and washed with N,N-dimethylformamide (100 ml). The filtrate was distilled and n-propanol (200 ml) was added to the solid obtained. The mixture was stirred for 4 hours at 55° C. to 60° C., filtered, washed with n-propanol (50 ml) and dried under vacuum at 45° C. to 50° C. to obtain the title compound.

Yield: 75.8%

We claim:

1. A process for the preparation of 3-(4-nitro-1-oxo-1,3-dihydro-2H-isoindol-2-yl)piperidine-2,6-dione of Formula II, 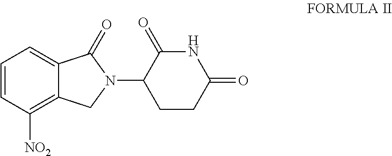
wherein the process comprises reacting methyl 2-bromomethyl-3-nitrobenzoate of Formula III 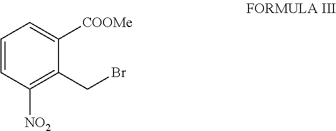
with 3-aminopiperidine-2,6-dione or its salt in the presence of an organic solvent at a temperature of about 50° C. or below to obtain 3-(4-nitro-1-oxo-1,3-dihydro-2H-isoindol-2-yl)piperidine-2,6-dione of Formula II.

2. A process for the preparation of lenalidomide, wherein the process comprises,
a) reacting methyl 2-bromomethyl-3-nitrobenzoate of Formula III 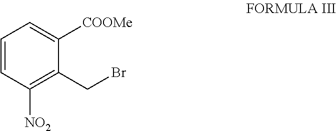
with 3-aminopiperidine-2,6-dione or its salt in the presence of an organic solvent at a temperature of about 50° C. or below to obtain 3-(4-nitro-1-oxo-1,3-dihydro-2H-isoindol-2-yl)piperidine-2,6-dione of Formula II, and 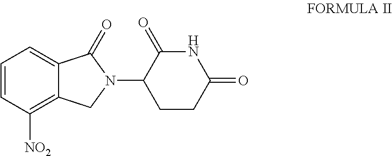
b) reducing 3-(4-nitro-1-oxo-1,3-dihydro-2H-isoindol-2-yl)piperidine-2,6-dione of Formula II to obtain lenalidomide.

3. A process according to claim 1 or 2, wherein the methyl 2-bromomethyl-3-nitrobenzoate of Formula III is reacted with 3-aminopiperidine-2,6-dione or its salt at a temperature of about 20° C. about 45° C.

4. A process according to claim 1 or 2, wherein the organic solvent comprises a water-miscible solvent.

5. A process according to claim 4, wherein the organic solvent comprises N,N-dimethylformamide, dimethylformamide, C₁₋₄ alkanol, C₃₋₆ ketone or acetonitrile, or a mixture thereof.

6. A process according to claim 1 or 2, wherein the 3-(4-nitro-1-oxo-1,3-dihydro-2H-isoindol-2-yl)piperidine-2,6-dione of Formula II has a purity of about 99.0% or above.

7. A process for the preparation of lenalidomide, wherein the process comprises:
a) reducing the 3-(4-nitro-1-oxo-1,3-dihydro-2H-isoindol-2-yl)piperidine-2,6-dione of Formula II in a solvent system comprising N,N-dimethylformamide 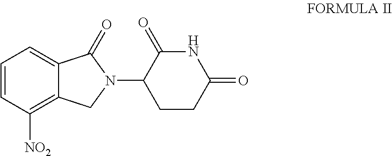
to obtain lenalidomide; and
b) isolating lenalidomide from the reaction mixture thereof.

8. A process according to claim 7, wherein the N,N-dimethylformamide is used as a single solvent or in combination with one or more water-miscible organic solvents.

9. A process according to claim 8, wherein the water-miscible organic solvent comprises methanol.

10. A process according to claim 7, wherein the solvent comprises a volume, which is about 2 times to about 50 times more than the weight of 3-(4-nitro-1-oxo-1,3-dihydro-2H-isoindol-2-yl)piperidine-2,6-dione of Formula II.

11. A process according to claim 7, wherein the lenalidomide has a purity of greater than about 99.8%.
